SEQUENCE LISTING (1 sequence)

SEQ ID NO: 1 - protein, 139 aa, Homo sapiens
  MISC_FEATURE (1)..(139): pro-EPIL sequence (UniProtKB/Swiss-Prot Accession no. Q14641)
MASLFRSYLPAIWLLLSQLLRESLAAELRGCGPRFGKHLLSYCPMPEKTFTTTPGGWLLESGRPKEMVSTSNNKDGQALGTTSEFIPNLSPELKKPLSEGQPSLKKIILSRKKRSGRHRFDPFCCEVICDDGTSVKLCT

(19) United States
(12) Patent Application Publication
(10) Pub. No.: US 2017/0122959 A1
(43) Pub. Date: May 4, 2017

(54) EARLY PLACENTA INSULIN-LIKE PEPTIDE (PRO-EPIL)

(71) Applicant: CÉZANNE S.A.S., Nimes Cedex 1 (FR)

(72) Inventors: Gaïané DEMIRDJIAN, Marseille (FR); Delphine ESPINASSE, Generac (FR); Nicolas BOURGOIN, Bernis (FR)

(21) Appl. No.: 15/311,169

(22) PCT Filed: May 18, 2015
(86) PCT No.: PCT/EP2015/060878

§ 371 Date: Nov. 14, 2016

(30) Foreign Application Priority Data
May 16, 2014 (EP) 14168715.2

(51) Int. Cl. G01N 33/68

(52) U.S. Cl. CPC G01N 33/689; G01N 2333/471; G01N 2800/387; G01N 2800/38

(57) ABSTRACT

The present invention relates to a method for the diagnosis, prognosis, risk assessment, risk stratification and/or therapy control of a prenatal disorder or condition in a pregnant female subject or the unborn fetus of said subject comprising the steps of: i) providing a sample of a bodily fluid of said subject; ii) determining the level of early placenta insulin-like peptide (pro-EPIL) or a fragment thereof in said sample; iii) comparing the determined level in the sample to a control level derived from subjects without said prenatal disorder or condition; wherein a deviation from said control level is indicative for said prenatal disorder or condition in said subject.

### BACKGROUND OF THE INVENTION

At least 130 million women give birth every year worldwide. About 15% of them experience a pregnancy related complication or illness.

Gestational diabetes mellitus (GDM) afflicts 3-7% of pregnancies in the United States (Berg, C. J., et al, Obstet Gynecol, 2009. 113(5): p. 1075-81) and 2-6% in Europe (Buckley B S., et al, Diabet Med, 2012 July; 29(7): 844-54) and is associated with unfavorable perinatal outcomes including fetal macrosomia, shoulder dystocia, cesarean-section, and neonatal hypoglycemia (ACOG Practice Bulletin. Number 30, September 2001 (replaces Technical Bulletin Number 200, December 1994). Gestational diabetes. Obstet Gynecol, 2001. 98(3): p. 525-38). Importantly, treatment of GDM has been shown to improve pregnancy outcomes (Crowther C et al, NEJM, 2005, 352:2477-2486). There are no tests at the present time for the prediction of GDM. Early diagnosis, i.e. before the onset of clinical symptoms, will allow clinicians to monitor at-risk patients closely and offer therapies characterized by a respective diet, physical exercises and/or administration of substances such as insulin and metformin that have been shown to be beneficial in this population (HAPO Study Cooperative Group. N Engl J Med 2008, 358: 1991-02; Rowan et al, N Engl J Med, 2008, 358:2003-2015).

In the United States and in some European countries, GDM is routinely detected at 24-28 weeks gestation by universal screening procedures. Presently, there is no universally agreed-upon gold standard test for the detection of GDM. The American Diabetes Association (ADA), the Canadian Society of Obstetricians and Gynecologists (SOGC), and the American College of Obstetrics and Gynecology (ACOG) recommend that all pregnant women be screened for GDM at about 28 weeks of gestation using a random 50 g glucose challenge test (GCT) regardless of fasting status, and testing blood glucose 1 hour later, followed by a diagnostic fasting 100 g 3-hour oral glucose tolerance test (OGTT) if their screening is positive. These tests are not 100% reliable; over 20% of women with a positive GCT will test positive with an OGTT (false positive high).

In many countries, screening methods for determining the risk of prenatal complications and/or fetal abnormalities have become routine to aid in treating and advising pregnant women. For example, in Europe, health care providers commonly screen for chromosomal abnormalities in the fetus using biochemical markers present in maternal blood. The combination of maternal age, the serological markers pregnancy associated plasma protein A (PAPP-A) and free-beta human chorionic gonadotropin (free β-hCG) and the ultrasound marker nuchal translucency (NT) thickness has been demonstrated to function in week 11⁺⁰-13⁺⁶ with a detection rate for Down syndrome of about 82 to 90% for a false positive rate of 5% (Malone et al. 2005. NEJM 353 (19): 2001-2010; Bindra et al. 2002. Ultrasound Obstet Gynecol 20: 219-225). Such screening is helpful for identifying women who have a sufficiently high risk to justify further diagnostic testing, which can be invasive and carry a risk for the fetus. However, this screening still fails to detect a significant number of Down syndrome cases and other aneuploidy affected pregnancies and diagnoses still 5% falsely positive.

Fetal growth restriction (FGR) is defined as a failure to achieve the endorsed growth potential. The diagnosis of fetal ‘smallness’ is currently performed on the basis of an estimated fetal weight (EFW) by ultrasounds below a given threshold, most commonly the 10^{th} centile (Figueras et al. 2014. Fetal Diagn Ther). Currently no routine screens have been adopted for early detection of FGR using maternal samples. The search for non-invasive, biomarkers that could predict the development or assist in the detection of this life-threatening pregnancy disorder is still of utmost importance. If the risk of fetal growth restriction could be detected earlier, better outcomes, including severity reduction and even recovery would be possible in many cases. During the pregnancy, at an early or later stage, a reliable risk assessment method of delivering a “small for gestational age” neonate or assessment of the presence of intrauterine growth restriction would decrease the potential for negative health of the baby.

Many studies have assessed the performance of first-trimester screening for FGR by combining different variables, such as maternal history and biochemical and biophysical markers. However, the detection rates achieved were not sufficiently high (Figueras et al. 2011. Am J Obstet Gynecol 204(4): 288-300).

Thus, there exists a need for accurate screening methods for prenatal complications and/or fetal abnormalities.

Human placenta is a source of several proteins and peptides active in the feto maternal adaptive responses (Petraglia et al. 1996. Endocr Rev 17:156-86). It is widely accepted that these placental factors have an impact on physiological and pathological disorders in pregnancy. Insulin and insulin-like growth factors belong to a family of polypeptides essential for the proper regulation of physiological processes such as energy metabolism, cell proliferation, development and differentiation.

The EPIL peptide is encoded by the insulin-like 4 gene (INSL4). This gene was identified by screening a cDNA library of first-trimester human placenta. INSL4 is highly expressed in early placenta and, with the exception of faint expression in normal uterine tissues, expression of INSL4 transcripts was not detected in any other normal tissue tested thus far (Chassin et al. 1995. Genomics 29: 465-70). The early placenta insulin-like peptide (EPIL) encoded by the insulin-like 4 gene is a member of the insulin related gene family comprising insulin, relaxin (RLX), insulin-like growth factors 1 and 2 (IGFI I and IGF II), Leydig insulin-like peptide (LEY I-L) encoded by the INSL3 gene and peptides encoded by INSL5 and INSL6 genes.

EPIL is a 139-amino-acid polypeptide which is synthesized as a preprohormone characterized by a signal peptide (underlined), a B-chain (italics), a connecting C-peptide (bold) and a terminal A-chain (SEQ-ID No. 1). In placenta, it was found that trophoblast cells translate INSL4 mRNAs into immunoreactive pro-EPIL peptides comprising the B-, C- and A-chains (Bellet et al. 1997. J Clin Endocrinol Metab 82: 3169-72). Initially, pro-EPIL peptide was detected in amniotic fluid and maternal serum during normal pregnancy, and the excretion pattern of pro-EPIL was similar to that of free β-hCG, suggesting common regulation pathways (Mock et al. 1999. J Clin Endocrinol Metab 84: 2253-6). One study reports that aneuploid pregnancies were characterized by changes in amniotic fluid concentration of pro-EPIL but not in maternal serum (Mock et al. 2000. J Clin Endocrinol Metab 85: 3941-4). Bruni et al. describes an increased level of pro-EPIL in samples of FGR pregnancy relative to control samples (Bruni et al. 2007. Am J Obstet Gynecol 2007; 197: 606.e1-606.e4).

The present inventors have surprisingly found that the level of pro-EPIL in maternal serum is a useful tool for accurate screening methods for prenatal complications and/or fetal abnormalities.

### SUMMARY OF THE INVENTION

The present invention relates to a method for the diagnosis, prognosis, risk assessment, risk stratification and/or therapy control of a prenatal disorder or condition in a pregnant female subject or the unborn fetus of said subject comprising the steps of:
i) providing a sample of a bodily fluid of said subject,
ii) determining the level of early placenta insulin-like peptide (pro-EPIL) or a fragment thereof in said sample,
iii) comparing the determined level in the sample to a control level derived from subjects without said prenatal disorder or condition;
   wherein a significant deviation from said control level is indicative for said prenatal disorder or condition in said subject.

The invention also pertains in one aspect to a method for determining the risk of a chromosomal abnormality, preferably aneuploidy, more preferably trisomy 21, trisomy 18, trisomy 13, most preferably trisomy 21, in a fetus, comprising:
a) determining the level of early placenta insulin-like peptide (pro-EPIL), placental growth factor (P1GF), (and optionally pregnancy-associated plasma protein A (PAPP-A)) and free human chorionic gonadotropin (free β-hCG) in one or more blood samples taken from a pregnant female subject;
b) optionally determining an ultrasound marker of said fetus, and
c) determining the risk of the chromosomal abnormality in the fetus using the measured levels of pro-EPIL, P1GF, (optionally PAPP-A), and free β-hCG.

Preferably, one or one or more ultrasound markers of the fetus are additionally determined, and the risk of the chromosomal abnormality in the fetus using the levels of pro-EPIL, P1GF, (optionally PAPP-A), free β-hCG, and the one or more ultrasound marker of the fetus is determined.

Moreover, the invention relates in a further aspect to a method for the diagnosis, prognosis, risk assessment, risk stratification and/or therapy control of a prenatal disorder or condition in a pregnant female subject or the unborn fetus of said subject comprising the steps of:
i) providing a sample of a bodily fluid of said subject,
ii) determining the level of early placenta insulin-like peptide (pro-EPIL) or a fragment thereof in said sample,
iii) comparing the determined level in the sample to a control level derived from subjects without said prenatal disorder or condition;
wherein a decreased level in the sample from the subject as compared to the control level is indicative for the prenatal disorder or condition.

The prenatal disorder or condition in the pregnant female subject may, for example, be selected from gestational diabetes mellitus, preterm birth, fetal growth restriction, the risk of delivery of a large for gestational age neonate and pre-eclampsia, preferably gestational diabetes mellitus.

### DESCRIPTION OF DRAWINGS

FIG. 1 shows the dose response curve for the pro-EPIL homogenous sandwich fluoroimmunoassay using Time Resolved Amplified Cryptate Emission (TRACE) technology of examples 2 and 3.

FIG. 2 shows a box and whisker plot of pro-EPIL concentrations in women with normal pregnancy.

FIG. 3 shows a box and whisker plot of pro-EPIL multiple of median (MoM) in women with normal and trisomy 21 pregnancy.

FIG. 4 shows a box and whisker plot of pro-EPIL multiple of median (MoM) in women with normal and gestational diabetes pregnancy.

FIG. 5 shows a box and whisker plot of pro-EPIL multiple of median (MoM) in women with normal pregnancy and fetal growth restriction (FGR) group.

The following examples and figures are used for a more detailed explanation of the invention, but do not limit the invention to said examples and figures.

### DETAILED DESCRIPTION

The present invention relates to a method for the diagnosis, prognosis, risk assessment, risk stratification and/or therapy control of a prenatal disorder or condition in a pregnant female subject or the unborn fetus of said subject. The method comprises the determination of the level of early placenta insulin-like peptide (pro-EPIL) or a fragment thereof in a sample of a bodily fluid of the pregnant female subject. Deviations from control levels of pro-EPIL are indicative for the prenatal disorder or condition in the pregnant female subject or the unborn fetus of said subject (i.e. including prenatal complications in the pregnant female and/or fetal abnormalities in the fetus). Both, higher and lower pro-EPIL levels in the sample as compared to a control level can be indicative for the prenatal disorder or condition, depending on the specific prenatal disorder or condition. The control level is derived from the pro-EPIL level in a sample of a healthy pregnant individual or much preferred samples of a group of healthy pregnant individuals, i.e. subjects without said prenatal disorder or condition. Hence, the pro-EPIL level in the sample of the subject to be diagnosed is compared to a healthy cohort of pregnant females, preferably pregnant females that are in the same trimester of pregnancy as the subject to be diagnosed.

Therefore the present invention relates to a method for the diagnosis, prognosis, risk assessment, risk stratification and/or therapy control of a prenatal disorder or condition in a pregnant female subject or the unborn fetus of said subject comprising the steps of:
i) providing a sample of a bodily fluid of said subject,
ii) determining the level of early placenta insulin-like peptide (pro-EPIL) or a fragment thereof in said sample,
iii) comparing the determined level in the sample to a control level derived from subjects without said prenatal disorder or condition;
   wherein a significant deviation from said control level is indicative for said prenatal disorder or condition in said subject.

A significant deviation in this context is a deviation of at least 5%, preferably at least 10%, more preferably at least 20% from the control level.

The invention further pertains in one aspect to a method for the diagnosis, prognosis, risk assessment, risk stratification and/or therapy control of a prenatal disorder or condition in a pregnant female subject or the unborn fetus of said subject comprising the steps of:
i) providing a sample of a bodily fluid of said subject,
ii) determining the level of early placenta insulin-like peptide (pro-EPIL) or a fragment thereof in said sample,
iii) comparing the determined level in the sample to a control level derived from subjects without said prenatal disorder or condition;
   wherein a decreased level in the sample from the subject as compared to the control level is indicative for the prenatal disorder or condition.

Similarly, the invention also relates to a method for the diagnosis, prognosis, risk assessment, risk stratification and/or therapy control of a prenatal disorder or condition in a pregnant female subject or the unborn fetus of said subject comprising the steps of:
i) providing a sample of a bodily fluid of said subject,
ii) determining the level of early placenta insulin-like peptide (pro-EPIL) or a fragment thereof in said sample,
iii) comparing the determined level in the sample to a control level derived from subjects without said prenatal disorder or condition;
   wherein an increased level in the sample from the subject as compared to the control level is indicative for the prenatal disorder or condition.

Preferably herein, the control level is a median of levels of subjects without said prenatal disorder or condition, and a deviation of >20% from said median is indicative of the prenatal disorder or condition.

Hence, the invention further relates to a method for diagnosing a prenatal disorder or condition in a pregnant female subject or the unborn fetus of said subject, wherein the level of early placenta insulin-like peptide (pro-EPIL) is determined in a sample from the subject to be diagnosed and wherein a level of pro-EPIL below a “multiple of the median” (MoM) of 0.8, 0.7, 0.6, 0.5, 0.4, 0.3 or 0.2 of the control group is indicative for the prenatal disorder or condition. The invention also relates to a method for diagnosing a prenatal disorder or condition in a pregnant female subject or the unborn fetus of said subject, wherein the level of early placenta insulin-like peptide (pro-EPIL) is determined in a sample from the subject to be diagnosed and wherein a level of pro-EPIL above a “multiple of the median” (MoM) of 1.2, 1.7, 2.0, 2.5 of the control group is indicative for the prenatal disorder or condition. A multiple of the median (MoM) is a measure of how far an individual test result deviates from the median of the control group. MoM is commonly used to report the results of medical screening tests, particularly where the results of the individual tests are highly variable. Concentrations of prenatal screening analytes change constantly throughout pregnancy. For example, AFP concentrations in maternal serum increase by about 15% per week during the most favorable time for detecting open neural tube defects (15-20^{th} week of gestation). Converting these values to a gestational age-specific median value (MoM) normalizes for this gestational age effect. A laboratory therefore may first obtain measures on sera obtained routinely from e.g. 300 to 500 women. Measurements are initially expressed in mass units (e.g. ng/ml) or international units (e.g. IU/ml). Weighted log-linear regression analysis is used to calculate an equation to determine median levels for the analyte in question for each gestational week. Each women's measurement is then divided by the median value for the appropriate gestational age resulting in a multiple of the median (MoM). The overall median value in a population of women is, by definition, 1.00 MoM. A “multiple of the median” indicates the factor applied to a median value, e.g. 0.90 means 90% of the median value. In the following exemplary threshold values will be discussed. However, depending on the desired specificity and/or sensitivity or the control group, other threshold values may be used. For example, suitable threshold values for gestational diabetes, at first and second trimester, are 5.3 ng/ml (corresponding to a MoM of 0.31) and 7.4 ng/ml (corresponding to a MoM of 0.22), respectively. A value above 34.4 ng/ml (corresponding to a MoM of 1.71) may be indicative for trisomy 21. Suitable threshold values for pregnancy with fetal growth restriction, at second and third trimester, are 19.4 ng/ml (corresponding to a MoM of 0.66) and 47.5 ng/ml (corresponding to a MoM of 0.63), respectively.

The prenatal disorder or condition may be a disorder or condition of the pregnant female subject or the unborn fetus of the subject. For example, the prenatal disorder or condition may be gestational diabetes mellitus in the pregnant female subject and associated disorders. Said associated disorders can be manifested in the pregnant female subject or the fetus. Said pregnant female associated disorders may be one or more selected from pregnancy induced hypertension, pre-eclampsia, preterm birth, cardiovascular diseases, metabolic syndrome and type 2 diabetes mellitus. Said fetus associated disorders may be one or more selected from stillbirth, macrosomia, hypoglycaemia, hyperbilirubinaemia, respiratory distress syndrome, shoulder dystocia, birth asphyxia, brachial plexus injury and prematurity. The prenatal disorder or condition may also relate to adverse pregnancy outcomes or fetal development such as fetal growth restriction or “large for gestational age”. The prenatal disorder or condition may also relate to adverse pregnancy outcomes such as pre-eclampsia or the risk of the female subject to give preterm birth. The prenatal disorder or condition may also relate to genetic disorders that are characterized by chromosomal abnormalities, e.g. aneuploidies including trisomy 13, trisomy 18 and trisomy 21.

Gestational diabetes mellitus refers to hyperglycemic conditions during pregnancy. High glucose levels can affect the baby in late pregnancy and cause babys with macrosomia.

Preterm Birth refers to a birth of a baby less than 37 weeks gestational age.

Fetal growth restriction refers to a fetus with intrauterine growth restriction that has not reached its determined potential size. Fetal weight is below the 10th percentile.

Large for gestational age refers to a fetus with a fetal weight, which is greater than the 90th percentile for gestational age.

The term “preeclampsia” includes a hypertensive, multi-system disorder of pregnant women, characterized by hypertension and proteinuria. The most common symptoms of preeclampsia are high blood pressure, increased protein in the urine, and swelling or edema of hands and face. In certain embodiments of the invention, preeclampsia is defined as hypertension (systolic and diastolic blood pressure of ≧140 and 90 mm Hg, respectively) and proteinuria (protein excretion of ≧300 mg in a 24 h urine collection, or a dipstick of ≧2+).

“Diagnosis” in the context of the present invention relates to the recognition and (early) detection of a disease or clinical condition in a subject and may also comprise differential diagnosis. Also the assessment of the severity of a disease or clinical condition may in certain embodiments be encompassed by the term “diagnosis”.

“Prognosis” relates to the prediction of an outcome or a specific risk for a subject suffering from a particular disease or clinical condition. This may include an estimation of the chance of recovery or the chance of an adverse outcome for said subject.

In the present invention, the terms “risk assessment” and “risk stratification” relate to the grouping of subjects into different risk groups according to their further prognosis. Risk stratification also relates to stratification for applying preventive and/or therapeutic measures.

The term therapy control in the context of the present invention refers to the monitoring and/or adjustment of a therapeutic treatment of said patient.

The term “screening” in the context of the present invention refers to a process of surveying a population, using a specific marker or markers and defined screening cut-off levels, to identify the individuals in the population at higher risk for a particular disorder. Screening is applicable to a population; diagnosis is applied at the individual patient level.

In the context of a prenatal disorder or condition that is associated with a decreased pro-EPIL level, a level of pro-EPIL in the sample of said subject that is below a multiple of the median (MoM) of 0.8 as compared to the control level is indicative for the prenatal disorder or condition and/or an increased risk of the subject or fetus to acquire the prenatal disorder or condition and/or an increased risk of an aggravation of the prenatal disorder or condition. Similarly, in the context of a prenatal disorder or condition that is associated with an increased pro-EPIL level, a level of pro-EPIL in the sample of said subject that is above a multiple of the median (MoM) of 1.2 as compared to the control level is indicative for the prenatal disorder or condition and/or an increased risk of the subject or fetus to acquire the prenatal disorder or condition and/or an increased risk of an aggravation of the prenatal disorder or condition.

Prenatal disorders or conditions in the pregnant female that are associated with a decreased level of pro-EPIL are, for example, gestational diabetes mellitus, preterm birth, fetal growth restriction, the risk of delivery of a large for gestational age neonate and pre-eclampsia. In a particularly preferred embodiment of the present invention, the prenatal disorder or condition in the pregnant female subject is gestational diabetes mellitus (GDM). In another particularly preferred embodiment the prenatal disorder or condition is fetal growth restriction (FGR).

Prenatal disorders or conditions in the unborn fetus of the pregnant female include for example chromosomal abnormalities, e.g. aneuploidy such as trisomy 13, trisomy 18, trisomy 21, Turner syndrome and triploidy. In a particularly preferred embodiment of the present invention, the prenatal disorder or condition is trisomy 13, trisomy 18 or trisomy 21, preferably trisomy 21.

In the context of the diagnostic, prognostic and risk assessment methods of the invention, further decisive parameters may be determined in addition to the pro-EPIL level. For example information relating to the age, weight and/or body mass index (BMI) may be additionally used for the assessment. Further parameters include the presence or former presence of other diseases or conditions in the subject. Moreover, other relevant biomarkers may be determined in the sample. Therefore, pro-EPIL may be part of a marker panel used in the diagnostic, prognostic and risk assessment methods of the invention. For example, when the disorder or condition is gestational diabetes, the further parameter may be blood glucose and the application of a glucose challenge test or an oral glucose tolerance test. For example, when the disorder or condition is aneuploidy, the further parameter may be one or more selected from the group consisting of pregnancy-associated plasma protein A (PAPP-A), free β-hCG, nuchal translucency (NT), maternal age, alpha-fetoprotein (AFP), placental growth factor (PlGF), fetal nasal bone, biparietal diameter/nasal bone ratio, human Chorionic Gonadotropin (hCG), inhibin A, unconjugated estriol 3 and cell free fetal DNA. For example, when the disorder or condition is fetal growth restriction, the further parameter may be one or more selected from the group consisting of PAPP-A, maternal history, PlGF, soluble fms-like tyrosine kinase 1 (sFlt1), AFP, free β-hCG, inhibin A, activin A, short-Endoglin (sEng), symphysis-fundal height, fetal biometry, head circumference, biparietal diameter, abdominal circumference, ultrasound markers (e.g. pulsatile-index, Umbilical artery Doppler, Middle cerebral artery doppler, Ductus venosus Doppler).

Herein, PlGF includes free PlGF and PlGF bound to multimeric complexes. PlGF therefore includes all PlGF isoforms unless stated otherwise.

The term “biomarker” (biological marker) relates to measurable and quantifiable biological parameters (e.g., specific enzyme concentration, specific hormone concentration, specific gene phenotype distribution in a population, presence of biological substances) which serve as indices for health- and physiology-related assessments, such as disease risk, psychiatric disorders, environmental exposure and its effects, disease diagnosis, metabolic processes, substance abuse, pregnancy, cell line development, epidemiologic studies, etc. Furthermore, a biomarker is defined as a characteristic that is objectively measured and evaluated as an indicator of normal biological processes, pathogenic processes, or pharmacologic responses to a therapeutic intervention. A biomarker may be measured on a biological sample (such as a blood, urine, or tissue test), it may be a recording obtained from a person (blood pressure, ECG, or Holter), or it may be an imaging test (Uteroplacental Doppler ultrasound, or nuchal translucency (Conde-Agudelo et al. 2004. Obstet Gynecol 104: 1367-1391; Bindra et al. 2002. Ultrasound Obstet Gynecol 20: 219-225)). Biomarkers can indicate a variety of health or disease characteristics, including the level or type of exposure to an environmental factor, genetic susceptibility, genetic responses to exposures, biomarkers of subclinical or clinical disease, or indicators of response to therapy. Thus, a simplistic way to think of biomarkers is as indicators of disease trait (risk factor or risk biomarker), disease state (preclinical or clinical), or disease rate (progression). Accordingly, biomarkers can be classified as antecedent biomarkers (identifying the risk of developing an illness), screening biomarkers (screening for subclinical disease), diagnostic biomarkers (recognizing overt disease), staging biomarkers (categorizing disease severity), or prognostic biomarkers (predicting future disease course, including recurrence and response to therapy, and monitoring efficacy of therapy). Biomarkers may also serve as surrogate end points. A surrogate end point is one that can be used as an outcome in clinical trials to evaluate safety and effectiveness of therapies in lieu of measurement of the true outcome of interest. The underlying principle is that alterations in the surrogate end point track closely with changes in the outcome of interest. Surrogate end points have the advantage that they may be gathered in a shorter time frame and with less expense than end points such as morbidity and mortality, which require large clinical trials for evaluation. Additional values of surrogate end points include the fact that they are closer to the exposure/intervention of interest and may be easier to relate causally than more distant clinical events. An important disadvantage of surrogate end points is that if clinical outcome of interest is influenced by numerous factors (in addition to the surrogate end point), residual confounding may reduce the validity of the surrogate end point. It has been suggested that the validity of a surrogate end point is greater if it can explain at least 50% of the effect of an exposure or intervention on the outcome of interest. For instance, a biomarker may be a protein, peptide or a nucleic acid molecule.

In the context of the methods of the present invention, different types of additional parameters can be distinguished: a) “low molecular weight” biomarkers such as glucose, b) “biochemical” biomarkers, i.e. biomolecules in the sample such as polypeptides, proteins and nucleic acids (particularly DNA), c) “biophysical” parameters e.g. information retrieved from imaging methods such as ultrasonic methods, d) other information such as the maternal age or maternal health history.

Additional “biochemical” biomarkers preferably relied on in the context of the present invention are PAPP-A, PlGF, sFlt1, AFP, free β-hCG, inhibin A, activin A, sEng, hCG and cell free fetal DNA.

Additional “biophysical” parameters preferably included in the context of the present invention are fetal symphysis-fundal height, fetal biometry, fetal head circumference, fetal biparietal diameter, fetal abdominal circumference, an ultrasound marker (e.g. selected from pulsatile-index, Umbilical artery Doppler, Middle cerebral artery doppler, Ductus venosus Doppler), nuchal translucency (NT), fetal nasal bone, fetal biparietal diameter/nasal bone ratio.

The term “sample” herein is a biological sample. “Sample” as used herein refers to a sample of bodily fluid for the purpose of diagnosis, prognosis, or evaluation of a subject of interest, such as a patient. A “patient” or “subject” for the purposes of the present invention includes both humans and other animals, particularly mammals, and other organisms. Thus, the methods are applicable to both human diagnostics and veterinary applications. In a preferred embodiment the patient is a mammal, and in the most preferred embodiment the patient or subject is a human.

Herein, the sample is a sample of a bodily fluid of the pregnant female subject. Preferred test samples include blood, serum, plasma, cerebrospinal fluid, amniotic fluid, urine, saliva, sputum, and pleural effusions. In addition, one of skill in the art would realize that some test samples would be more readily analyzed following a fractionation or purification procedure, for example, separation of whole blood into serum or plasma components.

Thus, in a preferred embodiment of the invention the sample is selected from the group comprising a blood sample, a serum sample, a plasma sample, a cerebrospinal fluid sample, a saliva sample and a urine sample or an extract of any of the aforementioned samples. Preferably, the sample is a blood sample, more preferably a serum sample or a plasma sample. Serum samples are the most preferred samples in the context of the present invention.

The term “reference sample” relates to a sample obtained from a subject or group of subjects who do not have a disease or disorder, and who do not develop the disease or disorder. Said subject or group of subjects represent the same gender and species as the subject being tested. The “control level” is derived from one or more “reference sample” and is preferably derived from subjects without a prenatal disorder or condition. It is preferred herein, that the “control group” is from the same trimester of pregnancy as the subject or even from the same gestational age (e.g. in terms of weeks of gestation). E.g. when the subject is in week 22-24 of gestation the control group may also be from week 22-24 of gestation.

“Plasma” in the context of the present invention is the virtually cell-free supernatant of blood containing anticoagulant obtained after centrifugation. Exemplary anticoagulants include calcium ion binding compounds such as EDTA or citrate and thrombin inhibitors such as heparinates or hirudin. Cell-free plasma can be obtained by centrifugation of the anticoagulated blood (e.g. citrated, EDTA or heparinized blood) for at least 15 minutes at 2000 to 3000 g.

“Serum” is the liquid fraction of whole blood that is collected after the blood is allowed to clot. When coagulated blood (clotted blood) is centrifuged serum can be obtained as supernatant. It does not contain fibrinogen, although some clotting factors.

The term “subject” as used herein refers to a living human or non-human female organism. Preferably herein the subject is a human subject that is pregnant within the first to third trimester, more preferably within the first trimester. For example, the subject is in week 11-13 of gestation. A “fetus” herein refers to a developing mammal after the embryonic stage and before birth. In humans, the fetal stage commences at the beginning of the ninth week of gestation.

The age of a pregnancy is called the “gestational age”. Typically, the initiation of pregnancy for the calculation of “gestational age” (e.g. in terms of trimesters or weeks of gestation) is the first day of the woman's last normal menstrual period. Pregnancies are typically divided into three stages called trimesters. According to typical definitions, the first trimester includes weeks 1 to 13, the second trimester includes weeks 14 to 26 and the third trimester includes the time from week 27 to birth. The method of the invention can in principal be carried out during all trimester of pregnancy. However, for certain diseases the method is preferably performed in specific phases of the pregnancy, e.g. the diagnosis of trisomy 21 is preferably performed in the first trimester, most preferably in weeks 10 to 13. On the other hand, the diagnosis of fetal growth restriction (FGR) using the method of the invention is preferably performed in the second and third trimesters.

The term “fragment” refers to smaller proteins or peptides derivable from larger proteins or peptides, which hence comprise a partial sequence of the larger protein or peptide. Said fragments are derivable from the larger proteins or peptides by saponification of one or more of its peptide bonds.

The term “level” in the context of the present invention relates to the concentration (preferably expressed as weight/volume; w/v) of marker peptides taken from a sample of a patient.

Determining the level of early placenta insulin-like peptide (pro-EPIL) or fragments thereof as well as other polypeptide-based biomarkers such as PlGF, PAPP-A, free β-hCG and sFlt1 herein is performed using a detection method and/or a diagnostic assay.

The preferred detection methods comprise immunoassays in various formats such as for instance radioimmunoassay (RIA), chemiluminescence- and fluorescence-immunoassays, enzyme immunoassay (EIA), Enzyme-linked immunoassays (ELISA), Luminex-based bead arrays, protein microarray assays, rapid test formats such as for instance immunochromatographic strip tests, and Selected/Multiple reaction monitoring (SRM/MRM).

The assays can be homogenous or heterogeneous assays, competitive and non-competitive assays. In a particularly preferred embodiment, the assay is in the form of a sandwich assay, which is a non-competitive immunoassay, wherein the molecule to be detected and/or quantified is bound to a first antibody and to a second antibody. The first antibody may be bound to a solid phase, e.g. a bead, a surface of a well or other container, a chip or a strip, and the second antibody is an antibody which is labeled, e.g. with a dye, with a radioisotope, or a reactive or catalytically active moiety. The amount of labeled antibody bound to the analyte is then measured by an appropriate method. The general composition and procedures involved with “sandwich assays” are well-established and known to the skilled person (The Immunoassay Handbook, Ed. David Wild, Elsevier LTD, Oxford; 3rd ed. (May 2005), ISBN-13: 978-0080445267; Hultschig C et al., Curr Opin Chem Biol. 2006 February; 10(1):4-10. PMID: 16376134, incorporated herein by reference).

In a particularly preferred embodiment, the assay comprises two capture molecules, preferably antibodies which are both present as dispersions in a liquid reaction mixture, wherein a first labelling component is attached to the first capture molecule, wherein said first labelling component is part of a labelling system based on fluorescence- or chemiluminescence-quenching or amplification, and a second labelling component of said marking system is attached to the second capture molecule, so that upon binding of both capture molecules to the analyte a measurable signal is generated that allows for the detection of the formed sandwich complexes in the solution comprising the sample.

Even more preferred, said labelling system comprises rare earth cryptates or rare earth chelates in combination with fluorescence dye or chemiluminescence dye, in particular a dye of the cyanine type.

In the context of the present invention, fluorescence based assays comprise the use of dyes, which may for instance be selected from the group comprising FAM (5- or 6-carboxyfluorescein), VIC, NED, Fluorescein, Fluoresceinisothiocyanate (FITC), IRD-700/800, Cyanine dyes, such as CY3, CY5, CY3.5, CY5.5, Cy7, Xanthen, 6-Carboxy-2′,4′,7′,4,7-hexachlorofluorescein (HEX), TET, 6-Carboxy-4′,5′-dichloro-2′,7′-dimethodyfluorescein (JOE), N,N,N′,N′-Tetramethyl-6-carboxyrhodamine (TAMRA), 6-Carboxy-X-rhodamine (ROX), 5-Carboxyrhodamine-6G (R6G5), 6-carboxyrhodamine-6G (RG6), Rhodamine, Rhodamine Green, Rhodamine Red, Rhodamine 110, BODIPY dyes, such as BODIPY TMR, Oregon Green, Coumarines such as Umbelliferone, Benzimides, such as Hoechst 33258; Phenanthridines, such as Texas Red, Yakima Yellow, Alexa Fluor, PET, Ethidiumbromide, Acridinium dyes, Carbazol dyes, Phenoxazine dyes, Porphyrine dyes, Polymethin dyes, and the like.

In the context of the present invention, chemiluminescence based assays comprise the use of dyes, based on the physical principles described for chemiluminescent materials in Kirk-Othmer, Encyclopedia of chemical technology, 4^{th} ed., executive editor, J. I. Kroschwitz; editor, M. Howe-Grant, John Wiley & Sons, 1993, vol. 15, p. 518-562, incorporated herein by reference, including citations on pages 551-562. Preferred chemiluminescent dyes are acridiniumesters. Pro-EPIL may for example be detected using fully automated sandwich immunoassay systems on the B.R.A.H.M.S KRYPTOR compact PLUS instrument (Thermo Scientific B.R.A.H.M.S GmbH, Hennigsdorf/Berlin, Germany). This random access analyzer employs the sensitive Time Resolved Amplified Cryptate Emmission (TRACE) technology, based on a non-radioactive-transfer between two fluorophores.

The sensitivity and specificity of a diagnostic and/or prognostic test depends on more than just the analytical “quality” of the test, they also depend on the definition of what constitutes an abnormal result. In practice, Receiver Operating Characteristic curves (ROC curves), are typically calculated by plotting the value of a variable versus its relative frequency in “normal” (i.e. apparently healthy individuals not having a prenatal disorder or condition) and “disease” populations. For any particular marker, a distribution of marker levels for subjects with and without a disease will likely overlap. Under such conditions, a test does not absolutely distinguish normal from disease with 100% accuracy, and the area of overlap indicates where the test cannot distinguish normal from disease. A threshold is selected, below which the test is considered to be abnormal and above which the test is considered to be normal. The area under the ROC curve is a measure of the probability that the perceived measurement will allow correct identification of a condition. ROC curves can be used even when test results do not necessarily give an accurate number. As long as one can rank results, one can create a ROC curve. For example, results of a test on “disease” samples might be ranked according to degree (e.g. 1=low, 2=normal, and 3=high). This ranking can be correlated to results in the “normal” population, and a ROC curve created. These methods are well known in the art. See, e.g., Hanley et al. 1982. Radiology 143: 29-36. Preferably, a threshold is selected to provide a ROC curve area of greater than about 0.5, more preferably greater than about 0.7, still more preferably greater than about 0.8, even more preferably greater than about 0.85, and most preferably greater than about 0.9. The term “about” in this context refers to +/−5% of a given measurement.

The horizontal axis of the ROC curve represents (1-specificity), which increases with the rate of false positives. The vertical axis of the curve represents sensitivity, which increases with the rate of true positives. Thus, for a particular cut-off selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

In other embodiments, a positive likelihood ratio, negative likelihood ratio, odds ratio, or hazard ratio is used as a measure of a test's ability to predict risk or diagnose a disorder or condition (“diseased group”). In the case of a positive likelihood ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group. In the case of a negative likelihood ratio, a value of 1 indicates that a negative result is equally likely among subjects in both the “diseased” and “control” groups; a value greater than 1 indicates that a negative result is more likely in the test group; and a value less than 1 indicates that a negative result is more likely in the control group.

In the case of an odds ratio, a value of 1 indicates that a positive result is equally likely among subjects in both the “diseased” and “control” groups; a value greater than 1 indicates that a positive result is more likely in the diseased group; and a value less than 1 indicates that a positive result is more likely in the control group.

In the case of a hazard ratio, a value of 1 indicates that the relative risk of an endpoint (e.g., death) is equal in both the “diseased” and “control” groups; a value greater than 1 indicates that the risk is greater in the diseased group; and a value less than 1 indicates that the risk is greater in the control group.

The skilled artisan will understand that associating a diagnostic or prognostic indicator, with a diagnosis or with a prognostic risk of a future clinical outcome is a statistical analysis. For example, a marker level of lower than X may signal that a patient is more likely to suffer from an adverse outcome than patients with a level more than or equal to X, as determined by a level of statistical significance. Additionally, a change in marker concentration from baseline levels may be reflective of patient prognosis, and the degree of change in marker level may be related to the severity of adverse events. Statistical significance is often determined by comparing two or more populations, and determining a confidence interval and/or a p value. See, e.g., Dowdy and Wearden, Statistics for Research, John Wiley & Sons, New York, 1983. Preferred confidence intervals of the invention are 90%, 95%, 97.5%, 98%, 99%, 99.5%, 99.9% and 99.99%, while preferred p values are 0.1, 0.05, 0.025, 0.02, 0.01, 0.005, 0.001, and 0.0001.

In a particular embodiment, the method of the invention is used for the risk assessment of a pregnant subject carrying a fetus with an aneuploidy such as trisomy 21. The risk assessment may be conducted by the determination of the level of pro-EPIL and at least one further parameter selected from the level of PlGF, PAPP-A and free human chorionic gonadotropin (free β-hCG). Additional other parameters such as maternal age, nuchal translucency (NT) and/or further additional biochemical, biophysical and maternal history parameters can be included.

In the context of the present invention, preferably combinations of different parameters including pro-EPIL and other biomarkers as well as biophysical markers such as nuchal translucency (NT) and additional information such as maternal age. These parameters can be combined into a score or algorithm that predicts the likelihood of the prenatal disorder or condition in the pregnant subject or the unborn fetus. For the risk assessment using more than one biomarker, the individual likelihood ratio (LR) for each biomarker may preferably be determined and multiplied together. Moreover, a correlation coefficient (r) is advantageously calculated that represents the correlation between the parameters. Example 3 shows r values representing correlations between parameters that are very useful and preferred for the risk assessment of an aneuploidy such as trisomy 21. Similarly, LR and correlation coefficients for other combinations and/or disorders/conditions can be calculated.

As described in the embodiment of Example 3, statistical analysis of a clinical population showed, that the combined measurement of pro-EPIL, PlGF, PAPP-A, free β-hCG with the determination of maternal age and with or without using an additional biophysical parameter such as nuchal translucency (NT), may preferably be conducted for the risk assessment of a pregnant subject carrying a fetus with an aneuploidy. A particular way of calculating the risk assessment of an aneuploidy such as trisomy 21 using maternal age, pro-EPIL, PlGF, PAPP-A, free β-hCG and nuchal translucency is explained in the following with reference to Example 3:

The maternal age accounts for a specific prevalence and a prior risk (Snijders et al. 1999, Ultrasound Obstet Gynecol 3 (3): 167-70). The given risk is expressed as odds and further multiplied by a LR (from the log Gaussian distributions) of pro-EPIL in trisomy 21 affected pregnancies and unaffected pregnancies. The unaffected distribution parameters are SD=0.2411, whereas trisomy 21 mean and SD are mean log pro-EPIL MoM=0.229 and SD=0.177. The final odds are converted back into a risk. For the combined detection of pro-EPIL with PlGF, PAPP-A and free β-hCG, correlation coefficients between the log MoM values in trisomy 21 and unaffected pregnancies are calculated. For unaffected pregnancies the correlation coefficients are with PlGF: 0.195, with PAPP-A 0.098, with free β-hCG 0.392. For pregnancies affected with trisomy 21 the correlation coefficients are with PlGF: −0.091, with PAPP-A 0.131, with free β-hCG −0.090.

Similar calculations can be performed for other disorders/conditions including aneuploidies such as trisomy 13 and trisomy 18.

As described in Example 3 and shown in Tables 1a and 1b, statistical analysis of a clinical population showed that the combined measurement of pro-EPIL, PlGF, PAPP-A, free β-hCG with the determination of maternal age and with or without using a biophysical parameter such as nuchal translucency, is a meaningful tool for the risk assessment of a pregnant subject carrying a fetus with an aneuploidy. For example, the determination of maternal age, free β-hCG, PlGF, pro-EPIL and nuchal translucency (NT) had a detection rate of 100% with a false positive rate of only 2.7% (at a cut-off of 1 in 250). Without using a biophysical parameter (i.e. NT in this example), the determination of maternal age, PAPP-A, free β-hCG, PlGF and pro-EPIL had a detection rate of 96.3% with a 9.6% false positive rate (cut off 1 in 250).

In the context of the present invention, the term “detection rate” is the percentage of affected individuals with a positive result. The term “false positive rate” is the percentage of unaffected individuals with a positive result.

Diagnosis of Fetal Growth Restriction (FGR)

The present invention relates in one aspect to a method for the diagnosis of fetal growth restriction (FGR) in a pregnant female subject. Hence, the invention pertains in a preferred embodiment to a method for the diagnosis of fetal growth restriction (FGR) in a pregnant female subject comprising the steps of:
i) providing a sample of a bodily fluid of said subject,
ii) determining the level of early placenta insulin-like peptide (pro-EPIL) or a fragment thereof in said sample,
iii) comparing the determined level in the sample to a control level derived from subjects without FGR;
   wherein a decreased level in the sample from the subject as compared to the control level is indicative for FGR.

Preferably in this aspect of the invention, additionally the level of placental growth factor (PlGF) in the sample of said subject is determined. A low PlGF level in the sample as compared to a control level is indicative for FGR. Yet another additional marker the level of which may be determined in this aspect of the invention is sFlt1. A high sFlt1 level in the sample as compared to a control level is indicative for FGR. The method of this aspect is preferably performed in the second or third trimester of the pregnancy.

Additional markers and parameters for the diagnosis of FGR that can be used in the method of this aspect are, for example: PAPP-A, maternal history, AFP, free β-hCG, hCG, inhibin-A, activin A, sEng, symphysis-fundal height (SFH), fetal biometry (femur length (FL), head circumference (HC), biparietal diameter (BPD), abdominal circumference (AC)) and ultrasound markers (Pulsatile-Index, Umbilical artery Doppler, Middle cerebral artery Doppler, Ductus venosus Doppler).

Diagnosis of Gestational Diabetes Mellitus (GDM)

The present invention relates in another aspect to a method for the diagnosis of gestational diabetes mellitus (GDM) in a pregnant female subject. Hence, the invention pertains in a preferred embodiment to a method for the diagnosis, prognosis, risk assessment, risk stratification and/or therapy control of gestational diabetes mellitus (GDM) in a pregnant female subject comprising the steps of:
i) providing a sample of a bodily fluid of said subject,
ii) determining the level of early placenta insulin-like peptide (pro-EPIL) or a fragment thereof in said sample,
iii) comparing the determined level in the sample to a control level derived from subjects without GDM;
   wherein a decreased level in the sample from the subject as compared to the control level is indicative for GDM.

Additional markers and parameters for the diagnosis of GDM that can be used in the method of this aspect are, for example: Follistatin-Like 3 (FSTL3), sex hormone-binding globulin (SHBG), Adiponectin, leptin, resistin, glycosylated fibronectin, placental lactogen, C-reactive protein (CRP), activin, myostatin, a specific cytokine, sFlt-1, and/or P1GF. The specific cytokine can be, e.g., an immune/hematopoietin, an interferon, a tumor necrosis factor (TNF)-related molecule or a chemokine. Examples include interleukin (IL)-6, IL-8, IL-lbeta, monocyte chemoattractant protein (MCP)-I or TNF-alpha, or any combination thereof.

Diagnosis of Aneuploidy

The present invention relates in yet another aspect to a method for the diagnosis and/or risk stratification of an aneuploidy such as trisomy 13, 18 and 21 (preferably trisomy 21) in the unborn fetus of a pregnant female subject. Hence, the invention relates in a preferred embodiment to method for the diagnosis and/or risk stratification of an aneuploidy, preferably trisomy 13, 18 and 21, most preferably trisomy 21, in an unborn fetus of said subject comprising the steps of:
i) providing a sample of a bodily fluid of said subject,
ii) determining the level of early placenta insulin-like peptide (pro-EPIL) or a fragment thereof in said sample,
iii) comparing the determined level in the sample to a control level derived from subjects without said aneuploidy;
   wherein an increased level in the sample from the subject as compared to the control level is indicative for the aneuploidy.

In the method of this aspect the subject is preferably in the first or second trimester, most preferably first trimester, of pregnancy. Moreover in this aspect of the invention, it is preferred that the maternal age is included in the assessment. Maternal age is known to be a risk factor for fetal aneuploidies (in particular trisomy 21). The older the pregnant female the more likely is an aneuploidy (particularly trisomy 21) in her fetus (Snijders et al. 1999, Ultrasound Obstet Gynecol 3 (3): 167-70).

It is further preferred in this aspect of the invention that pro-EPIL is determined in addition to further biochemical and/or biophysical markers. For instance, the level of one or more biomarkers selected from placental growth factor (PlGF), PAPP-A, sFlt1, AFP, free β-hCG, inhibin A, activin A, sEng, hCG, unconjugated estriol 3 and cell free fetal DNA is determined in the sample of said subject. Preferably, in addition to the level of pro-EPIL additionally the level of one or more biomarkers selected from placental growth factor (PlGF), PAPP-A and free β-hCG is determined in the sample of said subject, more preferably additionally the level of two or all biomarkers selected from placental growth factor (PlGF), PAPP-A and free β-hCG is determined in the sample of said subject. Hence, referred marker panels include:
a) PAPP-A & pro-EPIL
b) free β-hCG & pro-EPIL
c) PlGF & pro-EPIL
d) PAPP-A & free β-hCG & pro-EPIL
e) PAPP-A & PlGF & pro-EPIL
f) free β-hCG & PlGF & pro-EPIL
g) PAPP-A & free β-hCG & PlGF & pro-EPIL. A combination of PAPP-A & free β-hCG & PlGF and pro-EPIL is preferred. In trisomy 21, pro-EPIL and free β-hCG are increased as compared to a control level and PAPP-A and PlGF are decreased as compared to a control level.

In addition to pro-EPIL (and preferably additionally maternal age) or the above marker panels (and preferably additionally maternal age) a biophysical parameter, preferably an ultrasound marker such as nuchal translucency (NT) is included in the diagnosis/risk assessment. Fetal NT thickness correlates with the risk of trisomy 21 and other fetal aneuploidies (Nicolaides K. 2011, Prenat Diagn 31:7-15). In a particularly preferred embodiment of this aspect of the invention the level of PAPP-A & free β-hCG & PlGF and pro-EPIL are determined and compared to a control level and information from maternal age and fetal NT thickness is included in the diagnosis/assessment of the aneuploidy, preferably trisomy 21.

Preferably, in the method of this aspect at least
i) the level of proEPIL, free β-hCG and PlGF, or
ii) the level of proEPIL, free β-hCG, PAPP-A and PlGF, or
iii) the level of proEPIL, free β-hCG, PlGF and an ultrasound marker, preferably nuchal translucency, or
iv) the level of proEPIL, free β-hCG, PAPP-A, PlGF and an ultrasound marker, preferably nuchal translucency,
   is determined.

In the context of this aspect preferably a set of likelihood ratios based on the level of the biomarkers and/or the ultrasound marker is determined. Preferably, a Gaussian analysis is performed to determine the likelihood ratios.

Moreover, a final risk based on
a) a prior risk of the pregnant female for trisomy 13, trisomy 18 and trisomy 21 in an unborn fetus and,
b) said set of likelihood ratios may be determined.

The prior risk may be based on maternal history information such as maternal age. The prior risk (e.g. expressed as odds) may therefore for instance be derived from the maternal age-specific prevalence, and is multiplied by a likelihood ratio (LR) from the log Gaussian distributions of biochemical markers (and other markers as the case may be) in affected and unaffected pregnancies.

The invention also pertains in one aspect to a method for determining the risk of a chromosomal abnormality, preferably aneuploidy, more preferably trisomy 21, trisomy 18, trisomy 13, most preferably trisomy 21, in a fetus, comprising:
a) determining the level of early placenta insulin-like peptide (pro-EPIL), placental growth factor (P1GF), (and optionally pregnancy-associated plasma protein A (PAPP-A)) and free human chorionic gonadotropin (free β-hCG) in one or more blood samples taken from a pregnant female subject;
b) optionally determining an ultrasound marker of said fetus, and
c) determining the risk of the chromosomal abnormality in the fetus using the measured levels of pro-EPIL, P1GF, (optionally PAPP-A), and free β-hCG.

Preferably, one or one or more ultrasound markers of the fetus are additionally determined, and the risk of the chromosomal abnormality in the fetus using the levels of pro-EPIL, P1GF, (optionally PAPP-A), free β-hCG, and the one or more ultrasound marker of the fetus is determined. The ultrasound marker is preferably nuchal translucency. The additional determination of PAPP-A in this context is preferred.

In the method of this aspect, the level of at least one further biochemical marker selected from alpha-fetoprotein (AFP), inhibin A, hCG, unconjugated estriol 3 and cell free fetal DNA may be determined and the risk of the chromosomal abnormality in the fetus may be determined using the levels of pro-EPIL, P1GF, PAPP-A, free β-hCG, and the at least one additional biochemical marker.

Preferably in this aspect, the one or more biological samples are preferably taken from the pregnant subject in the first trimester or second trimester (preferably the first trimester) of pregnancy, preferably the one or more biological samples are taken from the pregnant individual within weeks 10 to 19 of pregnancy, more preferably within weeks 11 to 13 of pregnancy.

In one embodiment of this aspect, determining the risk comprises calculating a final risk based on the prior risk of the female subject of developing the chromosomal abnormality in a fetus and a set of likelihood ratios based on the amounts of pro-EPIL, P1GF, PAPP-A, and free β-hCG. Preferably, a multivariate Gaussian analysis is performed to determine the likelihood ratios. The method may further comprise using likelihood ratios for one or more maternal history parameters, at least preferably maternal age.

As outlined herein above, the method of the invention can be used for determining the risk that a pregnant individual is carrying a fetus having a chromosomal abnormality based on the levels of the biochemical markers and biophysical markers.

The present invention also relates to an apparatus for determining risk of a prenatal disorder or condition in a pregnant female subject or the unborn fetus of said subject, preferably a chromosomal abnormality in a fetus, the apparatus comprising: a data input means for inputting the amounts of pro-EPIL, P1GF, PAPP-A, and free β-hCG in one or more blood samples obtained from a pregnant individual; and a calculation means for determining the risk of a chromosomal abnormality in a fetus using the amounts of the pro-EPIL, P1GF, PAPP-A, and free β-hCG.

Preferably, the apparatus further comprises a data input means for inputting at least one of the amounts of alpha-fetoprotein (AFP), inhibin A, hCG, unconjugated estriol 3 and cell free fetal DNA in one or more blood samples obtained from the pregnant individual; and determining the risk of a chromosomal abnormality in a fetus using the amounts of at least one of the amounts of alpha-fetoprotein (AFP), inhibin A, hCG, unconjugated estriol 3 and cell free fetal DNA, and the amounts of pro-EPIL, P1GF, PAPP-A, and free β-hCG.

The invention also relates to medical profiles for the pregnant individual based on the method of the invention. For example, the invention relates to a medical profile for a pregnant female subject, comprising information for determining risk of a chromosomal abnormality in a fetus, wherein the information comprises the levels of pro-EPIL, P1GF, PAPP-A, and free β-hCG in one or more blood samples from the pregnant individual, and wherein the medical profile is stored on a computer-readable medium.

The present invention also relates to the use of an anti-pro-EPIL antibody in any of the methods according to the invention.

The appended examples and figures are used for a more detailed explanation of the invention, but do not limit the invention to said examples and figures.

All patent and non-patent references cited herein are hereby incorporated by reference in their entirety.

```
Sequence
SEQ-ID No. 1:
pro-EPIL sequence (UniProtKB/Swiss-Prot Accession
no. Q14641).
                10 20 30 40
MASLFRSYLP AIWLLLSQLL RESLAAELRG CGPRFGKHLL
                50 60 70 80
SYCPMPEKTF TTTPGGWLLE SGRPKEMVST SNNKDGQALG
                90 100 110 120
TTSEFIPNLS PELKKPLSEG QPSLKKIILS RKKRSGRHRF
              130
DPFCCEVICD DGTSVKLCT
```

### EXAMPLES

### Labelling of Antibodies

In the assay, monoclonal antibody EPIL 15 directed against EPIL C-chain 98-108 region (WO 2009/133088 A1) and monoclonal antibody EPIL02 directed against EPIL A-chain 125-137 region (WO 2009/133088 A1) were coupled to cyanine 5.5 (GE Healthcare UK Limited) and to Lumi4®-Tb (Lumiphore, Inc., Richmond, Canada), respectively. The coupling reactions were performed according to the manufacturer's prescribed coupling protocols.

### Example 1: Development of a Pro-EPIL Assay Using Two Monoclonal Antibodies

A homogenous sandwich fluoroimmunoassay using Time Resolved Amplified Cryptate Emission (TRACE) technology (Mathis, 1993. Clin Chem 39(9): 1953-9) was developed for the detection of pro-EPIL.

The stock Lumi4®-Tb-conjugated antibody and cyanine 5.5-conjugated antibody solution were diluted at 0.3 μg/mL and 5 μg/mL with assay buffer (100 mmol/L sodium phosphate pH 7, 0.1% bovine serum albumin free protease, 0.2 mg/mL mouse IgG), respectively, prior to use. The recombinant human pro-EPIL (Abnova, Taiwan, Ref. No: H00003641-P01) was diluted in normal human serum to give pro-EPIL standards. The immunoassay was performed by incubating 50 μL of samples/calibrators, 50 μL of cyanine 5.5-conjugated antibody solution and 50 μL of Lumi4®-Tb-conjugated antibody solution at 37° C. on B.R.A.H.M.S KRYPTOR compact PLUS instrument (Thermo Scientific B.R.A.H.M.S GmbH, Hennigsdorf/Berlin, Germany), according to the manufacturer's instructions. The reaction time of the assay was 59 min. The specific fluorescence (RFU) was measured by simultaneous dual wavelength measurement at 707 and 620 nm using a B.R.A.H.M.S KRYPTOR compact PLUS instrument.

### Example 2: Dose Response Curve of the Assay

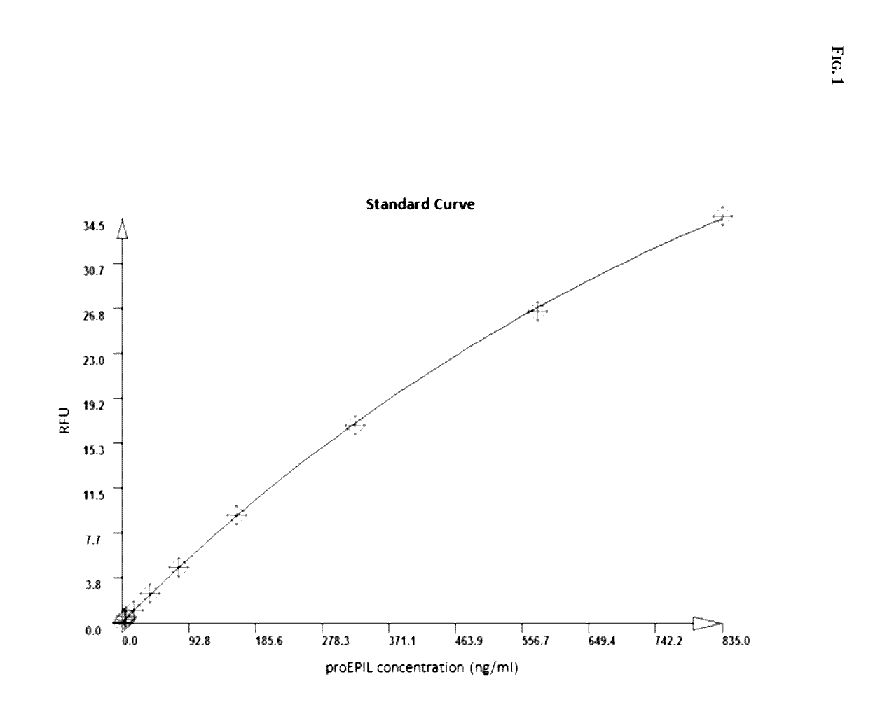

A dose response curve could be created by using the recombinant pro-EPIL as standard material in the monoclonal immunoassay as described above. A typical dose response curve is shown in FIG. 1.

### Example 3: Pro-EPIL in Prenatal Disorders

### Study Population

To determine whether pro-EPIL is a useful marker for pathological pregnancies, the homogenous sandwich fluoroimmunoassay for pro-EPIL described above was used to evaluate pro-EPIL in serum samples from 198 women who underwent normal pregnancies (first, second and third trimester), 11 pregnancies affected by gestational diabetes mellitus (GDM), 27 pregnancies affected by trisomy 21, and 43 pregnancies affected by intrauterine growth restriction (first, second and third trimester). The specific pro-EPIL assay was calibrated with recombinant human pro-EPIL.

Samples were taken at the routine first, second and third trimester hospital visit. All pregnant women signed a consent form, approved by different Hospital labs Ethics Committee.

### Measurements

pro-EPIL was detected using fully automated sandwich immunoassay systems on the B.R.A.H.M.S KRYPTOR compact PLUS instrument (Thermo Scientific B.R.A.H.M.S GmbH, Hennigsdorf/Berlin, Germany). This random access analyzer employs the sensitive Time Resolved Amplified Cryptate Emmission (TRACE) technology, based on a non-radioactive-transfer between two fluorophores. The automated assay for the detection of pro-EPIL is essentially based on the sandwich fluorescence assay using two monoclonal antibodies which are specifically binding to EPIL C-chain and EPIL A-chain and described above. The specific pro-EPIL assay was calibrated with recombinant human pro-EPIL. The BRAHMS pro-EPIL KRYPTOR assay had a measuring range of 2 to 835 ng/mL (FIG. 1). The limit of quantitation (functional sensitivity) was 4 ng/mL. The intra-assay and inter-assay variations were 1.2% and 5.7%, respectively at a pro-EPIL concentration of 25.5 ng/mL.

For determining the risk of gestational diabetes, pro-EPIL was measured in samples from pregnant women taken during the first and second trimester.

For determining the risk of chromosomal abnormality in the fetus the following parameters were measured in samples from pregnant women taken during the first trimester: i) PAPP-A, free β-hCG, ii) PAPP-A, free β-hCG, PlGF iii) PAPP-A, free β-hCG, pro-EPIL. The detection rate and false positive rate were determined using an algorithm combining these biomarkers.

For risk assessment analysis of the development of fetal growth restriction in pregnant women the following parameters were measured in samples from said subjects taken during the first, second and third trimester: PlGF, and pro-EPIL. The detection rate and false positive rate were determined using an algorithm combining these biomarkers.

### Statistical Analysis

Mathematical algorithms to determine risk of fetal aneuploidy are calculated by determining the medians for the normal population and the respective fetal aneuploidy population. PAPP-A, free β-hCG, PlGF and pro-EPIL are expressed in MoMs in order to standardized the findings. Box-whisker plot of pro-EPIL MoM of cases and control was created. Regression analysis was then used to determine the significance of association between pro-EPIL MoM with free β-hCG MoM, PAPP-A MoM and PlGF MoM. Similarly, the measured NT was expressed as a difference from the expected normal mean for gestation (delta value) and regression analysis was then used to determine the significance of association between pro-EPIL MoM and delta NT.

The MoM measured value of pro-EPIL, PlGF, PAPP-A, and/or free β-hCG can be corrected for ethnicity by dividing the MoM measured value of the biochemical marker (such as pro-EPIL, PlGF, PAPP-A, or free β-hCG) by the respective median value obtained from a group of pregnant women with unaffected pregnancies of the same ethnicity of the pregnant women. If desired, the MoM measured value of pro-EPIL, PlGF, PAPP-A, and/or free β-hCG is corrected for smoking by dividing the MoM measured value of the biochemical marker (such as pro-EPIL, PlGF, PAPP-A, or free β-hCG) by respective median value obtained from a group of pregnant women with unaffected pregnancies who smoke.

A prior risk (expressed as odds) is derived from the maternal age-specific prevalence, and is multiplied by an likelihood ratio (LR) from the log Gaussian distributions of biochemical markers in affected and unaffected pregnancies. For combinations of PAPP-A, free β-hCG, PlGF and pro-EPIL, correlation coefficients between log MoM values in affected and unaffected pregnancies are determined. The statistical process for carrying out the risk estimate is calculated by determining the medians for the unaffected pregnancies and the GDM and FGR groups. For each biochemical a MoM is calculated. Multivariate Gaussian analysis is then performed to determine likelihood ratios.

Mann-Whitney U test was applied to determine the significance of differences in the median MoM values in each outcome group to that in the controls.

### Results

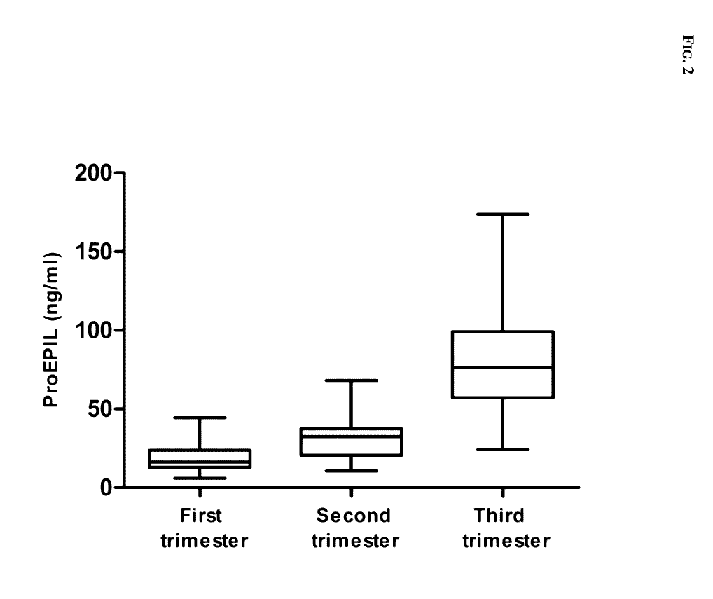

There was a correlation between the pro-EPIL concentration and gestational age (r=0.81; p<0.0001) in women with normal pregnancy. The median measured concentration of serum pro-EPIL was 16.2, 32.4 and 76.2 ng/ml at first, second and third trimester, respectively (FIG. 2).

In the normal group the mean log pro-EPIL MoM was −0.001 with a standard deviation (SD) of 0.241. There was a significant association between log pro-EPIL MoM and log free β-hCG MoM (r=0.392, p<0.0006), but not with log PAPP-A MoM (p=0.407), log PlGF MoM (p=0.099) and delta NT (p=0.855).

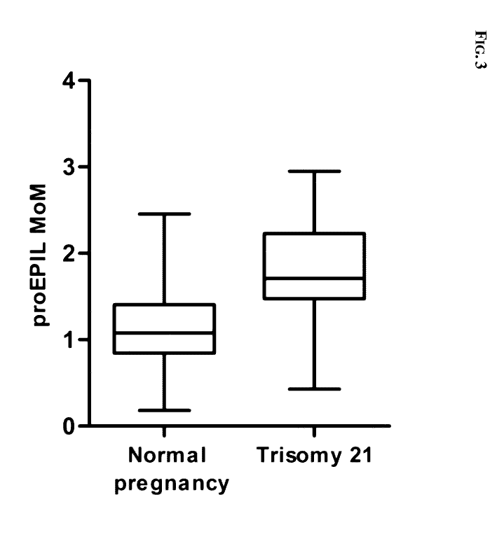

In the trisomy 21 group the median MoM of pro-EPIL (1.71 MoM) was significantly higher (p<0.0001) than in controls group (FIG. 3).

In trisomy 21 pregnancies, the mean log pro-EPIL MoM was 0.229 with a SD of 0.177. There was no significant association between log pro-EPIL MoM and log free β-hCG MoM (p=0.623), and log PAPP-A MoM (p=0.490), and log PlGF MoM (p=0.652) and delta NT (p=0.081).

Multiparameter analysis for risk assessment of trisomy 21 (for risk cut off 1 in 250) are given in Tables 1a and 1b. Including pro-EPIL in the risk calculation affects both, the detection rate and the false positive rate. There was an improvement of FPR when pro-EPIL is added to first trimester biochemistry (FPR: 9.6% and DR: 96.3%) (Table 1a). Moreover, one hundred percent of DR was reached when PAPP-A was replaced by pro-EPIL in the combined test (FPR: 2.7% and DR: 100%) (Table 1b).

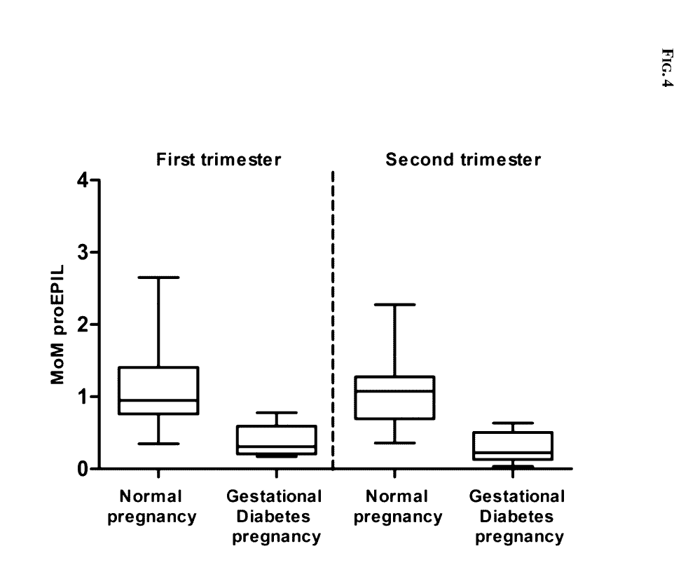

In GDM group, at first and second trimester, the medians MoM of pro-EPIL (0.31 and 0.22 MoM, respectively) were significantly lower (p=0.0017 and p=0.0005), than in normal pregnancy group (0.95 and 1.07 MoM) (FIG. 4).

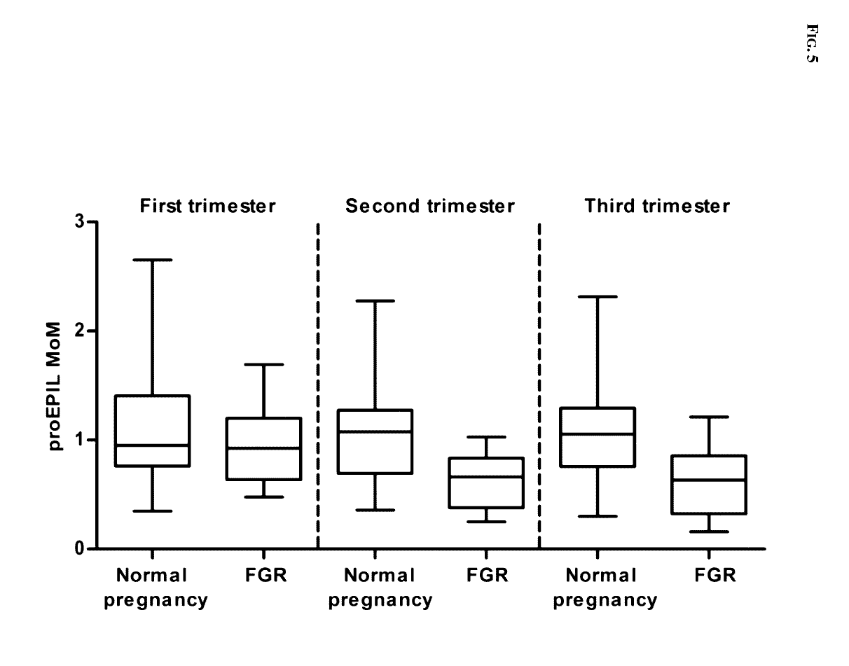

In the FGR group, at second and third trimester, the medians MoM of pro-EPIL (0.66 and 0.63 MoM, respectively) were significantly lower (p=0.0018 and p=0.0028) compared to controls group (1.07 and 1.05 MoM) (FIG. 5).

Multiparameter analysis for risk assessment of FGR showed (Table 2) that the determination of PlGF and pro-EPIL at second and third trimester was characterized by the highest detection rates (DR) of 57% and 67% respectively, compared to the DR after the measurement of PlGF (DR: 33% and 47%) or after the measurement of pro-EPIL (DR: 38% and 33%).

TABLE 1
Detection rate (DR) and false positive rates (FPR) for
risk assessment of aneuploidies using multiparameter
analysis with different algorithms (biochemical risks:
Table 1a; combined risks: Table 1b). Empirical
results for Trisomy 21 at first trimester

| Cut off (1 in 250) | |
|---|---|
| FPR (%) | DR (%) |

TABLE 1a

| | | |
|---|---|---|
| Maternal age, PAPP-A & free β-hCG | 16.4 | 96.3 |
| Maternal age, PAPP-A & PlGF | 16.4 | 92.6 |
| Maternal age, free β-hCG & PlGF | 12.3 | 92.6 |
| Maternal age, PAPP-A & pro-EPIL | 20.5 | 88.9 |
| Maternal age, free β-hCG & pro-EPIL | 16.4 | 88.9 |
| Maternal age, PlGF & pro-EPIL | 17.8 | 85.2 |
| Maternal age, PAPP-A & free β-hCG & PlGF | 12.3 | 96.3 |
| Maternal age, PAPP-A & free β-hCG & pro-EPIL | 16.4 | 96.3 |
| Maternal age, PAPP-A & PlGF & pro-EPIL | 15.1 | 88.9 |
| Maternal age, free β-hCG & PlGF & pro-EPIL | 13.7 | 96.3 |
| Maternal age, PAPP-A & free β-hCG & PlGF & | 9.6 | 96.3 |
| pro-EPIL | | |

Table 1b

| | | |
|---|---|---|
| Maternal age, PAPP-A, free β-hCG & NT | 4.1 | 96.3 |
| Maternal age, PAPP-A, PlGF & NT | 5.5 | 96.3 |
| Maternal age, free β-hCG, PlGF & NT | 4.1 | 96.3 |
| Maternal age, PAPP-A, pro-EPIL & NT | 12.3 | 96.3 |
| Maternal age, free β-hCG, pro-EPIL & NT | 4.1 | 100 |
| Maternal age, PlGF, pro-EPIL & NT | 5.5 | 92.6 |
| Maternal age, PAPP-A, free β-hCG, PlGF & NT | 5.5 | 96.3 |
| Maternal age, PAPP-A, free β-hCG, pro-EPIL & NT | 6.8 | 100 |
| Maternal age, PAPP-A, PlGF, pro-EPIL & NT | 8.2 | 92.6 |
| Maternal age, free β-hCG, PlGF, pro-EPIL & NT | 2.7 | 100 |
| Maternal age, PAPP-A, free β-hCG, PlGF, | 5.5 | 100 |
| pro-EPIL & NT | | |

TABLE 2
Detection rate (DR) for risk assessment of pregnancy with fetal growth
restriction using multiparameter analysis with different algorithms.

| | DR (%) for fixed False positive rate | |
|---|---|---|
| | (FPR)10% | |
| FGR | 2^{nd} trimester | 3^{rd} trimester |
| PlGF | 33 | 47 |
| pro-EPIL | 38 | 33 |
| PlGF and pro-EPIL | 57 | 67 |

1. A method for the diagnosis, prognosis, risk assessment, risk stratification and/or therapy control of a prenatal disorder or condition in a pregnant female subject or the unborn fetus of said subject comprising:
i) providing a sample of a bodily fluid of said subject,
ii) determining the level of early placenta insulin-like peptide (pro-EPIL) or a fragment thereof in said sample,
iii) comparing the determined level in the sample to a control level derived from subjects without said prenatal disorder or condition;
wherein an increased level in the sample from the subject as compared to the control level is indicative for the prenatal disorder or condition.

2. The method of claim 1, wherein a level of pro-EPIL in the sample of said subject that is above a multiple of the median (MoM) of 1.2 as compared to the control level is indicative for the prenatal disorder or condition and/or an increased risk of the subject or fetus to acquire the prenatal disorder or condition and/or an increased risk of an aggravation of the prenatal disorder or condition.

3. The method of claim 1, wherein the prenatal disorder or condition in the unborn fetus of the pregnant female subject is selected from trisomy 13, trisomy 18 and trisomy 21, optionally trisomy 21.

4. The method of claim 3, wherein said subject is in the first or second trimester of pregnancy.

5. The method of claim 1, wherein additionally the level of one or more biomarkers selected from placental growth factor (PlGF), PAPP-A, sFlt1, alpha-fetoprotein (AFP), free β-hCG, inhibin A, activin A, short-Endoglin (sEng), hCG, unconjugated estriol 3 and cell free fetal DNA is determined in a sample of said subject.

6. The method of claim 3, wherein additionally the level of one or more, optionally all, biomarkers selected from placental growth factor (PlGF), PAPP-A and free β-hCG is determined in a sample of said subject.

7. The method of claim 3, wherein additionally an ultrasound marker, optionally nuchal translucency, is determined.

8. The method of claim 3, wherein
i) the level of proEPIL, free β-hCG and PlGF, or
ii) the level of proEPIL, free β-hCG, PAPP-A and PlGF, or
iii) the level of proEPIL, free β-hCG, PlGF and an ultrasound marker, optionally nuchal translucency, or
iv) the level of proEPIL, free β-hCG, PAPP-A, PlGF and an ultrasound marker, optionally nuchal translucency,
is determined.

9. The method of claim 6, wherein a set of likelihood ratios based on the level of the biomarkers and/or the ultrasound marker is determined.

10. The method of claim 9, wherein a final risk based on
a) a prior risk of the pregnant female for trisomy 13, trisomy 18 and trisomy 21 in the unborn fetus and,
b) said set of likelihood ratios,
is determined.

11. The method of claim 9, wherein a Gaussian analysis is performed to determine the likelihood ratios.

12. A method for determining the risk of a chromosomal abnormality, optionally trisomy 21, trisomy 18, trisomy 13, in a fetus, comprising:
a) determining the level of early placenta insulin-like peptide (pro-EPIL), placental growth factor (P1GF) and free human chorionic gonadotropin (free β-hCG) in one or more blood samples taken from a pregnant female subject;
b) optionally determining an ultrasound marker, optionally nuchal translucency, of said fetus, and
c) determining the risk of the chromosomal abnormality in the fetus using the measured levels of pro-EPIL, P1GF and free β-hCG.

13. The method of claim 12, wherein determining the risk comprises calculating a final risk based on the prior risk of developing the chromosomal abnormality and a set of likelihood ratios based on the levels of pro-EPIL, P1GF, and free β-hCG.

14. The method according to claim 12, comprising:
a) determining the level of early placenta insulin-like peptide (pro-EPIL), placental growth factor (P1GF), pregnancy-associated plasma protein A (PAPP-A) and free human chorionic gonadotropin (free β-hCG) in one or more blood samples taken from a pregnant female subject;
b) optionally determining an ultrasound marker, optionally nuchal translucency, of said fetus, and
c) determining the risk of the chromosomal abnormality in the fetus using the measured levels of pro-EPIL, P1GF, PAPP-A, and free β-hCG.

15. The method of claim 14, wherein determining the risk comprises calculating a final risk based on the prior risk of developing the chromosomal abnormality and a set of likelihood ratios based on the levels of pro-EPIL, P1GF, PAPP-A, and free β-hCG.

16. A method for the diagnosis, prognosis, risk assessment, risk stratification and/or therapy control of a prenatal disorder or condition in a pregnant female subject or the unborn fetus of said subject comprising:
i) providing a sample of a bodily fluid of said subject,
ii) determining the level of early placenta insulin-like peptide (pro-EPIL) or a fragment thereof in said sample,
iii) comparing the determined level in the sample to a control level derived from subjects without said prenatal disorder or condition;
wherein a decreased level in the sample from the subject as compared to the control level is indicative for the prenatal disorder or condition.

17. The method of claim 16, wherein a level of pro-EPIL in the sample of said subject that is below a multiple of the median (MoM) of 0.8 as compared to the control level is indicative for the prenatal disorder or condition and/or an increased risk of the subject or fetus to acquire the prenatal disorder or condition and/or an increased risk of an aggravation of the prenatal disorder or condition.

18. The method of claim 16, wherein the prenatal disorder or condition in the pregnant female subject is selected from gestational diabetes mellitus, preterm birth, fetal growth restriction, the risk of delivery of a large for gestational age neonate and pre-eclampsia, optionally gestational diabetes mellitus.

19. The method of claim 16, wherein the prenatal disorder or condition is fetal growth restriction and wherein additionally the level of placental growth factor (PlGF) in the sample of said subject is determined.

20. The method of claim 19, wherein said subject is in the second or third trimester of pregnancy.
